(19) United States
(12) Patent Application Publication
(10) Pub. No.: US 2023/0210502 A1
(43) Pub. Date: Jul. 6, 2023

(54) A SAMPLING DEVICE

(71) Applicant: VITALOGRAPH (IRELAND) LTD., Enis, Co. CLare (IE)

(72) Inventors: Frank Keane, Limerick (IE); Colum Dunne, Co. Tipperary (IE)

(21) Appl. No.: 17/921,720

(22) PCT Filed: Apr. 28, 2021
(86) PCT No.: PCT/EP2021/061179

§ 371 Date: Oct. 27, 2022

(30) Foreign Application Priority Data
Apr. 30, 2020 (EP) 20172550.4

(51) Int. Cl. A61B 10/00; A61B 5/097

(52) U.S. Cl. CPC A61B 10/00; A61B 5/097; A61B 2010/0087

(57) ABSTRACT

An air sampling device (1) for collecting airborne microorganisms comprising an air inlet piece (2), an air outlet piece (3) and a filter chamber (4) for receiving a filter (5) between the air inlet piece (2) and the air outlet piece (3) wherein the sampling device (1) comprises an openable fastener (6) to provide ease of access to the filter chamber (4).

### INTRODUCTION

This invention relates to a sampling device for collecting airborne microorganisms for subsequent analysis. More particularly, the invention relates to an air sampling device adapted to collect exhaled airborne microorganisms from a patient for subsequent analysis.

### BACKGROUND OF THE INVENTION

Respiratory infections are very common in the community and contribute to the burden of illness for patients with chronic respiratory diseases, including acute exacerbations. This leads frequently to attendance at clinics for treatment and, often, use of antimicrobials such as antibiotics. ^{[1]}

Traditional sampling methods for respiratory infections are invasive and problematic to repeat. Depending on severity of the illness, and whether the illness is deemed chronic or acute, specimen collection for diagnosis can include antral/sinus washouts, bronchoalveolar lavage (BAL) or bronchoalveolar washings/brushings/zo aspirates, cough swabs, endotracheal tips, pleural aspirates/chest drain fluids, sputum, endotracheal or tracheal aspirates, nasopharyngeal aspirates/secretions, induced sputum or transbronchial/lung biopsy samples.

These sampling methods require skilled personnel with appropriate protection/isolation facilities and subsequent diagnosis may require microbiology, molecular or immunological laboratory processing before definitive results are available. ^{[2, 3]}

Commonly, many specimen collection techniques from the upper respiratory tract include oropharyngeal or nasopharyngeal swabs and nasopharyngeal washings or aspirates while lower respiratory tract samples are commonly collected via sputum that is induced or spontaneously expectorated. Bronchoalveolar lavage (BAL) is also sometimes used. Such nasopharyngeal techniques and BAL can be uncomfortable for patients while BAL is invasive and requires hospitalisation. ^{[4, 5]}

Where viral infections are suspected, e.g. coronaviruses such as SARs-CoV-2 (Covid-19), once retrieved, liquid virus transport medium (VTM) must also be employed to contain/maintain the sample before analysis e.g. by RNA extraction. However, when epidemics arise, reagents such as VTM can be in short supply thereby hindering testing for the virus. In addition, once the initial phase of an infection with a virus such as Covid-19 has passed, and the virus is in the lower airways, swabs may show a negative result as the swab is unable to retrieve sample from the lower airways while sample from the upper airways may test negative—essentially a false positive result for the patient.

US 2018/0164283 describes an air sampling device in which gelatine membrane filters are employed and a vacuum pump is required to sample the air. However, the air sampling device is not adapted for exhaled breath sampling and is not a dual function device suitable for use in breath sampling and capable of preventing cross-contamination protection with medical respiratory devices. US 2020/0109630 describes a breath sampling device for detecting drug substances. However, the device is a complex device and is not a sealed device adapted for use in safely catching and detecting infectious microorganisms and is also not suitable for use in preventing cross-contamination in medical devices. The device also lacks an openable fastener for ease of access to the filter. US 20180306775 also relates principally to a drug testing device which is also complex and lacks a dual functionality as a sampling device and cross contamination prevention mouthpiece. The device also appears to lack an openable fastener. US 2010/0159575 is a multipart apparatus for sampling pathogens in exhaled air. However, the apparatus is complex and lacks openable and closable fasteners for ease of access to filters. IE S67150 describes a filter unit for respiratory medical devices. However, the filter unit is not an exhaled air sampling device.

Accordingly, a need exists for improved sampling devices for respiratory infections that are easy to use and that are not invasive or uncomfortable for patients.

An object of the invention is to overcome at least some of the problems of the prior art.

### SUMMARY OF THE INVENTION

In its broadest sense the invention relates to a sampling device for collecting airborne microorganisms comprising:
an air inlet piece;
an air outlet piece and
a filter chamber for receiving a filter between the air inlet piece and the air outlet piece wherein the sampling device comprises an openable fastener to provide access to the filter chamber.

According to the invention there is provided a patient exhaled air sampling device for collecting airborne microorganisms comprising:
an air inlet piece for passive or pumped collection of a breath sample;
an air outlet piece removably attached to the air inlet piece to define a filter chamber between the air inlet piece and the air outlet piece for receiving an electrostatic filter and an air tight seal about the filter chamber wherein the sampling device comprises an openable fastener, movable between an open and closed position, between the air inlet piece and the air outlet piece to provide access to the filter chamber and the air inlet piece coacts with the air outlet piece to define a filter mount at the filter chamber.

Preferably, the fastener is formed between the air inlet piece and the air outlet piece. Suitably, the fastener comprises at least one clip. Advantageously, the at least one clip is a bendable clip.

Optionally, the fastener comprises a fastener wall depending from an inner face of the air inlet piece and a complementary oppositely disposed fastener wall depending from an inner face of the air outlet piece.

In any embodiment, the air tight seal is an interference fit seal defined by the fastener walls.

Optionally, the airtight seal is a crush seal. Preferably, the crush seal is formed by the coacting air inlet and outlet pieces.

Advantageously, the crush seal is formed with the fastener in the closed position.

Preferably, the crush seal comprises a peripheral crush rib on an internal face of the air inlet piece.

In any embodiment, the device further comprises a release to facilitate opening of the fastener.

Optionally, the release comprises a slot to provide access to the fastener.

In any embodiment, the release comprises a push-off between the air inlet piece and the air outlet piece.

In any embodiment, the filter mount comprises a first flange on an inner face of the air inlet piece and a complementary second flange on an inner face of the air outlet piece.

Preferably, the filter mount comprises a peripheral wall on an inner face of the air inlet piece and a complementary peripheral wall on an inner face of the air outlet piece.

Advantageously, the filter mount comprises a clamping bar to clamp the filter in the filter mount.

In any embodiment, the filter mount comprises at least one filter retainer for holding the filter in the filter mount. Preferably, the filter retainer comprises a retaining protrusion or pip. Suitably, the retaining protrusion or pip is provided on the air inlet piece or the air outlet piece.

In a preferred embodiment, the sampling device is a dual function device suitable for use as a mouthpiece on a respiratory medical device.

The invention also extends to a sampling device further comprising an electrostatic filter in the filter chamber.

Optionally, the sampling device further comprises a tamper evident mechanism and/or an anti-tamper locking mechanism.

Optionally, the sampling device further comprises removable caps for the air inlet piece and the air outlet piece.

In a preferred embodiment of the invention, the sampling device is a patient exhaled air sampling device for collecting exhaled airborne microorganisms which is also suitable for use as a mouthpiece on a respiratory medical device.

In another embodiment, the invention also extends to a method of diagnosing a microorganism caused respiratory infection in a human or animal patient comprising passively sampling an exhalation breath from the patient with a sampling device as hereinbefore defined and opening the sampling device at the openable fastener on the sampling device to remove the filter from the filter chamber for analysis.

Preferably, the method further comprises the steps of extracting microorganisms from the filter and analysing the extracted microorganisms.

Optionally, the analysis step comprises a PCR amplification step.

In any embodiment, the analysis step comprises DNA/RNA and/or immunological analysis. In other embodiments, the analysis step comprises culturing bacteria, viruses and/or fungi on culture media.

In one embodiment, the respiratory infection is a viral infection such as a SARs-CoV-2 (Covid-19) infection.

In a further embodiment, the invention also extends to a method of sampling the exhaled breath of a human or animal patient to detect a respiratory infection comprising sampling an exhalation breath from the patient with a sampling device as hereinbefore defined and opening the sampling device at an openable fastener on the sampling device to remove the filter from the filter chamber for analysis.

Preferably, the method further comprises the steps of extracting microorganisms from the filter and analysing the extracted microorganisms.

Optionally, the analysis step comprises a PCR amplification step.

In any embodiment, the analysis step comprises DNA/RNA and/or immunological analysis. In other embodiments, the analysis step comprises culturing bacteria, viruses and/or fungi on culture media.

In one embodiment, the respiratory infection is a viral infection such as a SARs-CoV-2 (Covid-19) infection.

In any embodiment, the sampling device forms the mouthpiece on a respiratory medical device.

The sampling device of the invention therefore collects microorganisms from the exhaled breath of subjects suspected of having respiratory illness, particularly viral respiratory infections such as SARs-CoV-2 (Covid-19). The sampling device can be employed for sampling the breath of humans or animals as required. The sampling device retrieves microorganism from the upper and lower respiratory tracts so that the incidence of false negatives is reduced. Accordingly, the sampling device of the invention is particularly useful for sampling purposes where aerosol transmission is a confirmed mechanism of disease spread, especially for viruses including influenza and Covid-19. ^{[6]}

The sampling device of the invention is also simple in construction and can be disassembled via its fastener and releases without requiring the use of specialised tools to provide ease of access to filters contained within the filter chamber.

Importantly, the sampling device of the invention has a dual function in that it can be employed as a patient exhaled air sampling device and also as mouthpiece on a respiratory medical device in which a patient exhales into the device e.g. lung function testing apparatus. The sampling device of the invention can also be used to analyse for and detect microorganisms that have been introduced into respiratory medical devices inadvertently (including but not limited to spirometers).

The preferred electrostatically-charged filter employed in the sampling device of the invention is capable of collecting microorganisms from air that passes through the filter while the airflow can be exhaled from patients as described above or be environmental air if desired.

The sampling device can be a moulded unit capable of bi-directional airflow and the filter can be removed easily for subsequent analysis.

Before passage of air and subsequent to specimen collection, the caps can be placed on the inlet and outlet of the housing unit to reduce risk of contamination of the filter material.

The sampling device of the invention is suitable for the detection of all airborne viruses, bacteria, fungi and other pathogens. In addition, the bacteria/viruses, once on the filter, do not have to remain “alive” or viable, but simply remain attached to the filter material so that the desired analytical techniques can then be used for detection e.g. PCR can be used to amplify the DNA/RNA as required and detect it. Accordingly, fewer reagents such as VTM that can be in short supply are required. The bacteria/viruses/fungi can also be cultured on media if desired.

The microorganisms collected using the sampling device of the invention can be viable or non-living.

In general, the collected microorganisms (bacteria, fungi or viruses) are sources of nucleic acid (DNA or RNA) which can be extracted from the viable or non-living microorganisms and then utilised for molecular biology assays employing techniques such as PCR, immunological assays or culturing techniques. The sampling device can therefore be employed as a diagnostic tool for human or animal use as required.

Due to its simplicity, the sampling device can be employed by a patient without the assistance of medical personnel. Accordingly, physical contact or proximity of the tester to the subject can be reduced or eliminated (e.g. where a patient provides a sample themselves at home).

The sampling device of the invention can also be employed as a component in diagnostic or environmental monitoring systems. The sampling device can also be employed in the assessment of potential inadvertent contamination of medical devices such as respiratory medical devices, by passing air through the devices and the sampling device of the invention and collecting samples for laboratory analysis—a positive result indicating a need for enhanced hygiene and/or cleaning. Accordingly, the sampling device can aid in a reduction in accidental transmission of respiratory infectious agents between users of the devices.

### BRIEF DESCRIPTION OF THE DRAWINGS

The invention will now be described, by way of example only, with reference to the accompanying drawings in which:

FIG. 1 is a perspective view from above and one side of a first embodiment of a sampling device of the invention for collecting airborne microorganisms having a square profile which is particularly suitable for use in collecting and retaining exhaled microorganisms for subsequent analysis;

FIG. 2 is an exploded view of the sampling device of FIG. 1 with the air inlet piece and the air outlet piece separated from the filter;

FIG. 3 is a plan view of the sampling device from the inlet piece side;

FIG. 4 is a side elevation of the sampling device;

FIG. 5 is a cross-sectional view along the line IV-IV of FIG. 4;

FIG. 6 is an enlarged view of the crush rib seal and a disassemblable mounting clip defined between the air inlet piece and air outlet piece to form a seal between the air inlet piece and the air outlet piece and to facilitate removal of the filter from the sampling device for analysis;

FIG. 7 is a perspective view from above and one side of a second embodiment of a sampling device of the invention having a round profile for collecting airborne microorganisms in which the air inlet piece and the air outlet piece meet at a seal formed by an interference fit;

FIG. 8 is an exploded view of the sampling device of FIG. 7 with the air inlet piece and the air outlet piece separated from the filter;

FIG. 9 is a plan view of the sampling device from the inlet piece side;

FIG. 10 is a side elevation of the sampling device;

FIG. 11 is a cross-sectional view along the line XI-XI of FIG. 4;

FIG. 12 is an enlarged view of the interference fit seal and fastener;

FIG. 13 is a side elevation of a third embodiment of the invention similar to the embodiments of FIGS. 1 to 12 but in which the filter mount includes a filter clamping bar and a pip-like retainer to retain the filter in the filter mount;

FIG. 14 is a cross-sectional view along the line XIV-XIV of FIG. 13, and

FIG. 15 is an enlarged view of the filter mount and fastener of the device showing the filter clamping bar and filter retaining pip or protrusion of the filter mount.

### DETAILED DESCRIPTION OF THE INVENTION

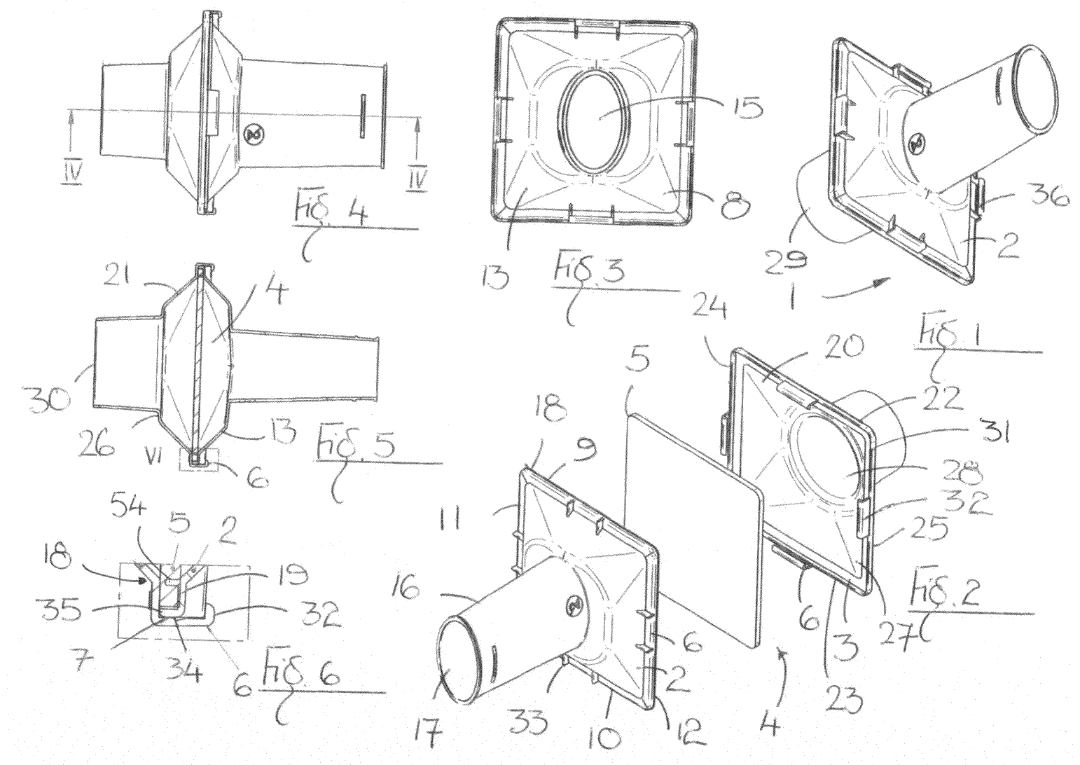

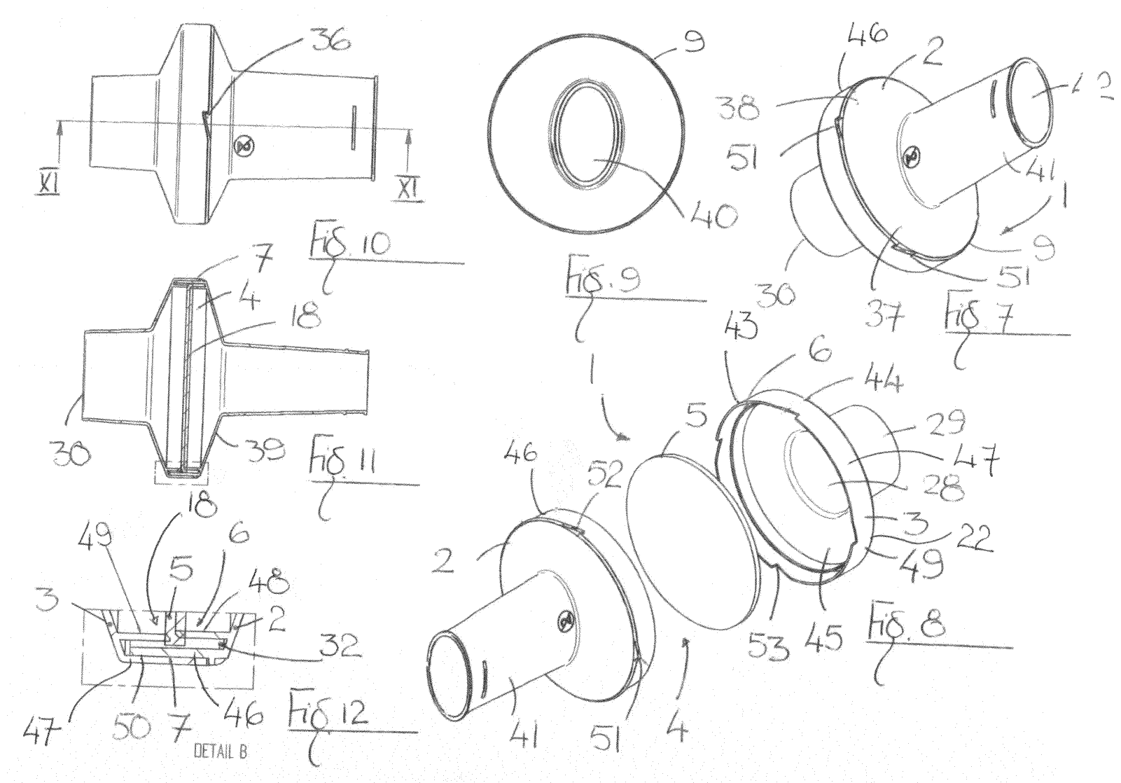

FIGS. 1 to 6 show a first embodiment of a sampling device 1 adapted to collect airborne microorganisms for subsequent analysis. In the following description, the sampling device is a patient exhaled air sampling device which is described with reference to the direct sampling of exhaled microorganisms from a patient. However, as will be appreciated by those skilled in the art, the sampling device 1 can also be used more generally for the collection of airborne microorganisms for subsequent analysis.

As shown in the drawings, the sampling device 1 is made up of an air-inlet piece 2 for receiving air into the sampling device 1, an air outlet piece 3 in fluid communication with the air inlet piece 2 for discharging air from the sampling device 1, a filter chamber 4 defined between the air inlet piece 2 and the air outlet piece 3 and an air filter 5 disposed in the filter chamber 4 for entrapping airborne microorganisms contained within air received through the air inlet piece The air sampling device 1 is disassemblable so that the air filter 5 can be easily removed from the air sampling device to analyse any microorganisms on the filter 5. More particularly, the air sampling device is disassemblable at an openable fastener 6, movable between an open and closed position, on the sampling device 1 to provide access to the filter chamber 4 to remove the filter 5 from the filter chamber 4 for analysis. As shall be explained more fully below, in the present embodiment, the fastener 6 is defined between the air inlet piece 2 and the air outlet piece 3. The sampling device 1 is also provided with an airtight fluid seal 7 to sealably attach the air inlet piece 2 to the air outlet piece 3 about the filter chamber 4.

The air inlet piece 2 is generally square in construction having a first square housing plate 8 defined by an upper first rim-like edge 9, an oppositely disposed second lower rim-like edge 10, a first rim-like side edge 11 and a second oppositely disposed rim-like side edge 12. The first housing plate 8 also defines an outer face 13 and an inner face 14 and is provided with a centrally located air inlet opening 15 having an outwardly extending annular mouthpiece 16 terminating at a mouthpiece opening 17 for receiving exhaled air from a patient into the sampling device 1.

The air sampling device 1 is further provided with a filter mount 18 to support the filter 5 in the filter chamber 4. In the present embodiment, the filter mount 18 is made up, in part, of a first peripheral flange 19 defined on the inner face 14 of the first housing plate 8 towards the edges 9, 10, 11, 12.

The air outlet piece 3 is complementary in shape and construction with the air inlet piece 2 and is made up of second complementary housing plate 20 which in combination with the first housing plate 8 defines a sampling device housing 21. The second complementary housing plate 20 is therefore also made up of an upper first rim-like edge 22, an oppositely disposed second lower rim-like edge 23, a first rim-like side edge 24 and a second oppositely disposed rim-like side edge 25. The edges 9, 10, 11, 12 of the air inlet piece 2 and the corresponding edges 22,23,24,25 of the air outlet piece 3 therefore meet to form the sampling device housing 21. The second complementary housing plate 20 also defines an outer face 26 and inner face 27 and is provided with an air outlet opening 28 for discharging air from the sampling device 1 into an outlet tube 29 on the outer face 26 which terminates at an outlet opening 30.

The second housing plate 20 is also provided with a second peripheral flange 31 complementary with the first peripheral flange 19 to complete the filter mount 18 so that the filter 5 is supported between the first and second peripheral flanges 19, 20 i.e. the air inlet piece 2 coacts with the air outlet piece 3 to define the filter mount 18 at the filter chamber 4. In addition, the filter mount 18 includes a clamping bar 54 to further securely clamp the filter in the filter mount 18. In the present embodiment, the clamping bar 54 is made up of an elongate wall-like structure extending outwards from the first peripheral flange 19.

The openable fastener 6 is formed between the air inlet piece 2 and the air outlet piece 3 at the edges 9,10,11,12,22,23,24,25 and is made up of four clips 32, with each clip 32 being centrally located along the edges 22, 23, 24, 25 of the second housing plate 20, which are engageable with the opposite edges 9, 10, 11, 12 13 on the first housing plate 8—the second housing plate 20 being sized so that the rim-like edges 22, 23, 24, 25 of the second housing plate 20 overlap the rim-like edges 9, 10, 11, 12 of the first housing plate 8. The rim-like edges 9, 10, 11, 12 of the first housing plate 8 are also provided with pairs of spaced apart guides 33 to assist in positioning the clips 32. Each clip 32 is also scored with an indent 34 formed to assist in allowing the bending of the clips 32 to open the clips 32 and provide access to the filter chamber 4. Furthermore, each clip 32 can be provided with a release 36 to facilitate opening of the clips 32. In the present embodiment, the clips 32 are shaped and sized to provide the release 36 in the form of a slot 36 to provide access to the underside of the clips 32 (e.g. with a digit or other item) to lift and release the clips 32.

In the present embodiment, the air seal 7 between the air inlet piece 2 and the air outlet piece 3 is a crush seal 7 formed about the filter chamber 4 by a crush rib 35 peripherally formed towards the edges 9, 10, 11, 12 of the inner face 27 of the first housing plate 8 of the air inlet piece 2. Accordingly, upon fastening of the air inlet piece 2 to the air outlet piece 3 by the fastener 6, the crush rib 35 is compressed to form the air seal 7.

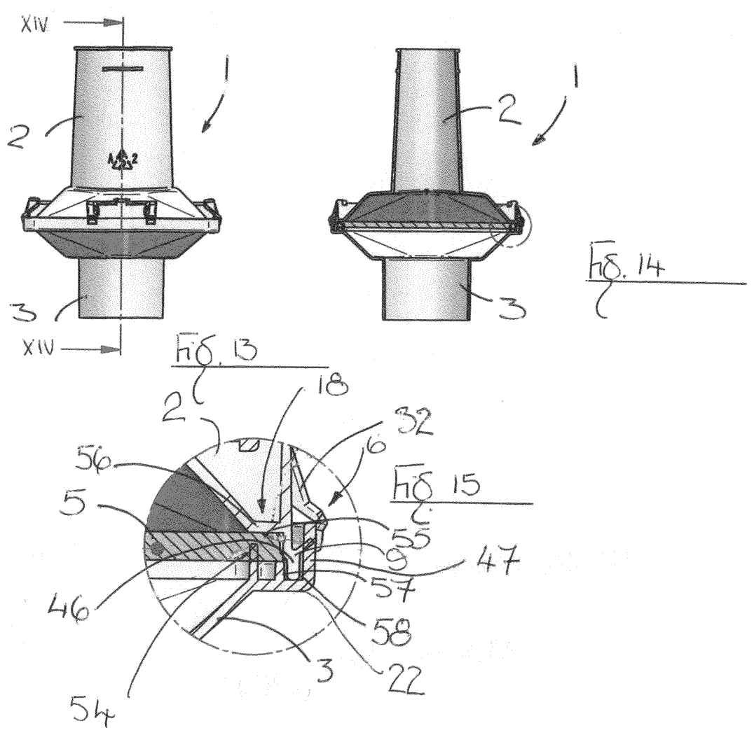

FIGS. 7 to 12 show a second embodiment of an air sampling device 1 of the invention functionally similar to the air sampling device of FIGS. 1 to 6 but having a generally round profile. Like numerals indicate like parts.

As shown in the drawings, the air inlet piece 2 of the sampling device 1 is made up of a first generally round housing plate 37 with a single round outer edge 9 and having an outer face 38 and an inner face 39 provided with an air inlet opening 40 in fluid communication with a mouthpiece 41 having a mouthpiece opening 42 for receiving air to be sampled. Similarly, the air outlet piece 3 is made up of a second complementary round housing plate 43 also having a single round outer edge 22 and an outer face 44 and an inner face 45 provided with an air outlet opening 28 for discharging air from the sampling device 1 into an outlet tube 29 on the outer face 26 which terminates at an outlet opening 30.

In the present embodiment, the fastener 6 is a single fastener 6 defined by a continuous, peripheral and circular fastener wall 46 depending from the inner face 39 of the first round housing plate 37 and a complementary oppositely disposed continuous, peripheral and circular fastener wall 47 depending from the inner face 45 of the second complementary round housing plate 43.

The seal 7 of the present embodiment is an interference fit seal 7 also formed by the peripheral and circular fastener walls 46,47. Accordingly, the peripheral and circular fastener walls 46,47 are sized and shaped to enjoy an interference fit 50 when the air inlet piece 2 is fastened to the air outlet piece 3 by the fastener 6 to create the seal 7 In this arrangement, the peripheral skirt-like wall 47 overlies or overlaps the peripheral skirt-like wall 46 when the air inlet piece 2 is attached to the air outlet piece 3.

In the present embodiment, the release 36 is provided in the form of at least one push-off 51 between the air inlet piece 2 and the air outlet piece 3 so that the air inlet piece 2 and the air outlet piece 3 can be urged apart thus overcoming the fastener 6 and seal 7 to provide access to the filter chamber 4. More particularly, the sampling device 1 is provided with four spaced apart push-offs 51 each made up of a peripheral protrusion 52 on the outer edge of the first round housing plate 37 of the air inlet piece 2 and a complementary push-off notch 53 defined on the skirt-like peripheral wall 49 of the second round housing plate 43 of the air outlet piece 3. Accordingly, upon counter clockwise rotation of the air inlet piece 2 with respect to the air outlet piece 3, the protrusions 52 on the air inlet piece 2 travel beneath skirt-like peripheral wall 49 to push off the air outlet piece 3.

The filter mount 18 of the present embodiment is made up of a peripheral circumferential wall 48 on the air inlet piece 2 disposed inwards of and spaced apart from the peripheral fastener wall 46 and an opposite disposed but offset peripheral circumferential wall 49 on the air outlet piece 3.

FIGS. 13 to 15 show another embodiment of the invention similar to the embodiment described in FIGS. 7 to 12 and like numerals indicate like parts. However, in the present embodiment, the filter mount 18 is made up of a filter clamping bar 54 as described above which is formed by a continuous upstanding peripheral wall on the outlet piece 3 adjacent the outer edge 22. In addition, the filter mount includes at least one filter retainer 55 on the air inlet piece 2 opposite but offset with respect to the clamping bar 54 to further retain the filter in place between the air inlet piece 2 and air outlet piece 3. In the present embodiment, the filter retainer 55 is in the form of a series of spaced apart circumferential protrusions or pips 55 disposed towards the edge 9 of the air inlet piece 2. The protrusions or pips 55 therefore coact with the clamping bar 54 to assist in securing the filter 5 in place In the present embodiment, the air inlet piece 2 is also provided with a peripheral flange 56 disposed towards the edge 9 and the air outlet piece 3 with an oppositely disposed upstanding wall 57 defining the filter mount 18 between which the filter 5 is supported. In addition, as shown in the drawing, the skirt-like wall 46 of the air inlet piece 2 is received in a channel 58 defined between the upstanding wall 57 and the peripheral skirt-like wall 47 to form an interference fit.

In use, the sampling device 1 can be employed as a standalone sampling device for sampling exhaled breath of subjects or can be attached, via the inlet piece 2, to a respiratory measurement device such as a spirometer thus serving as a dual function device.

Where the sampling device 1 is employed as a single use standalone respiratory device 1, the sampling device 1 with a filter 5 in the filter chamber 4 is presented to a subject who breathes into the mouthpiece 16 so that any microorganisms present in the subject's breath are entrapped in the filter 5. The filter 5 can then be removed by opening the fastener 6 and accessing the filter 5. Any microorganisms present on the filter 5 can then be extracted from the filter 5 and subjected to analytical techniques as required e.g. PCR followed by genetic analyses. As indicated above, the bacteria/viruses/fungi can also be cultured on media if desired.

The sampling device 1 of the invention can be used for airborne sampling with a passive airflow or with airflow mediated by a pump as required.

The sampling device 1 can also include removable sealing caps for the air inlet opening 15 and the air outlet opening 28 of the air inlet piece 2 and the air outlet piece 3 respectively which can be employed before and after use to reduce the risk of contamination of the filter 5.

The sampling device 1 can be a single use sampling device 1 which can be disposed of following use or, if desired, a multi-use device where the sampling device 1 is sterilised and re-used with a replacement filter 5.

Due to its simplicity, the sampling device 1 can be employed by a patient without the assistance of medical personnel. For example, the sampling device 1 could be mailed to a patient who would remove the sealing caps and follow instructions provided with the sampling device 1 for its use e.g. breathe through the sampling device 1 5-6 times, then re-insert the caps and place the sealed sampling device 1 in a return container for return mailing. The return container can have an anti-microbial coating to improve safety and prevent infection transmission.

A particularly preferred filter 5 suitable for use with the present invention is an electrostatically charged filter such as the Technostat (Trade Mark) filter available from Vitalograph which exhibits an efficiency in excess of 99.9999%. However, if desired, other filter types could be employed with the sampling device of the invention.

In another embodiment of the invention, the sampling device 1 is provided with tamper evident mechanism (e.g. bands/seals) and/or an anti-tamper locking mechanism to indicate if the device has been opened.

The sampling device 1 can also include marking on the filter 5 to indicate which side is the patient side i.e. which side is the microorganism contaminated side. In another embodiment, the sampling device 1 can also be provided with a bite lip on the mouthpiece 16 to allow a patient to grip the sampling device 1 firmly.

The sampling device 1 can be formed from any suitable materials including plastics materials such as polypropylene. Alternatively, the sampling device 1 can be formed from biodegradable and/or recyclable materials if desired.

### REFERENCES

^{[1]} O'Connor, R., O'Doherty, J., O'Regan, A., O'Neill, A., McMahon, A., Dunne, C. (2019) Medical management of acute upper respiratory infections in an urban primary care out-of-hours facility: cross-sectional study of patient presentations and expectations. BMJ Open 15;9(2):e025396. doi: 10.1136/bmjopen-2018-025396
^{[2]} Campbell, S., and Forbes, B. A. (2011) Proceedings of Camp Clin Micro 2011. The Clinical Microbiology Laboratory in the Diagnosis of Lower Respiratory Tract Infections. J. Clin. Microbiol. 49 (9 Suppl) S30-33
^{[3]} Skodvin, B., Wathne, J. S., Lindemann, P. C. et al. (2019) Use of microbiology tests in the era of increasing AMR rates—a multicentre hospital cohort study. Antimicrob Resist Infect Control 8, 28 https://doi.org/10.1186/s13756-019-0480-z
^{[4]0} Blaschke, A. J. et al (2011) Non-invasive sample collection for respiratory virus testing by multiplex PCR. J. Clin. Virol. 52 210-4
^{[5]0} Heikkinen, T., Marttila, J., Salmi, A. A. and Ruuskanen, O. (2002) Nasal swab versus nasopharyngeal aspirate for isolation of respiratory viruses. J. Clin. Microbiol. 40 4337-9
^{[6]} van Doremalen, N., Bushmaker, T., Morris, D. H., et al. (2020) Aerosol and surface stability of SARS-CoV-2 as compared with SARS-CoV-1. New Eng J Med 382;16 DOI: 10.1056/NEJMc2004973

1. A patient exhaled air sampling device for collecting airborne microorganisms comprising:
an air inlet piece for passive or pumped collection of a breath sample;
an air outlet piece removably attached to the air inlet piece to define a filter chamber between the air inlet piece and the air outlet piece for receiving an electrostatic filter and an air tight seal about the filter chamber wherein the sampling device comprises an openable fastener, movable between an open and closed position, between the air inlet piece and the air outlet piece to provide access to the filter chamber and the air inlet piece coacts with the air outlet piece to define a filter mount at the filter chamber.

2. A patient exhaled air sampling device as claimed in claim 1 wherein the fastener is formed between the air inlet piece and the air outlet piece.

3. A patient exhaled air sampling device as claimed in claim 2 wherein the fastener comprises at least one clip.

4. A patient exhaled air sampling device as claimed in claim 3 wherein the at least one clip is a bendable clip.

5. A patient exhaled air sampling device as claimed in claim 1 wherein the fastener comprises a fastener wall depending from an inner face of the air inlet piece and a complementary oppositely disposed fastener wall depending from an inner face of the air outlet piece.

6. A patient exhaled air sampling device as claimed in claim 5 wherein the air tight seal is an interference fit seal defined by the fastener walls.

7. A patient exhaled air sampling device as claimed in any of claims 1 to 4 wherein the airtight seal is a crush seal.

8. A patient exhaled air sampling device as claimed in claim 7 wherein the crush seal is formed by the coacting air inlet and outlet pieces.

9. A patient exhaled air sampling device as clamed in claim 8 wherein the crush seal is formed with the fastener in the closed position.

10. A patient exhaled air sampling device as claimed in any of claims 7 to 9 wherein the crush seal comprises a peripheral crush rib on an internal face of the air inlet piece.

11. A patient exhaled air sampling device as claimed in any of claims 1 to 10 further comprising a release to facilitate opening of the fastener.

12. A patient exhaled air sampling device as claimed in claim 11 wherein the release comprises a slot to provide access to the fastener.

13. A patient exhaled air sampling device as claimed in claim 12 wherein the release comprises a push-off between the air inlet piece and the air outlet piece.

14. A patient exhaled air sampling device as claimed in any of claims 1 to 13 wherein the filter mount comprises a first flange on an inner face of the air inlet piece and a complementary second flange on an inner face of the air outlet piece.

15. A patient exhaled air sampling device as claimed in any of claims 1 to 14 wherein the filter mount comprises a peripheral wall on an inner face of the air inlet piece and a complementary peripheral wall on an inner face of the air outlet piece.

16. A patient exhaled air sampling device as claimed in any of claims 1 to 15 wherein the filter mount comprises a clamping bar to clamp the filter in the filter mount.

17. A patient exhaled air sampling device as claimed in any of claims 1 to 16 wherein the filter mount comprises at least one filter retainer for holding the filter in the filter mount.

18. A patient exhaled air sampling device as claimed in claim 17 wherein the filter retainer comprises a retaining protrusion or pip.

19. A patient exhaled air sampling device as claimed in claim 18 wherein the retaining protrusion or pip is provided on the air inlet piece or the air outlet piece.

20. A patient exhaled air sampling device as claimed in any of claims 1 to 19 wherein the sampling device is a dual function device suitable for use as a mouthpiece on a respiratory medical device.

21. A patient exhaled air sampling device as claimed in any of claims 1 to 20 further comprising an electrostatic filter in the filter chamber.

22. A patient exhaled air sampling device as claimed in any of claims 1 to 21 further comprising a tamper evident mechanism and/or an anti-tamper locking mechanism.

23. A patient exhaled air sampling device as claimed in any of claims 1 to 22 further comprising removable caps for the air inlet piece and the air outlet piece.

24. A method of diagnosing a microorganism caused respiratory infection in a human or animal patient comprising:
passively sampling an exhalation breath from the patient with a sampling device as claimed in any of claims 1 to 24.
and opening the sampling device at the openable fastener on the sampling device to remove the filter from the filter chamber for analysis.

25. A method as claimed in claim 24 further comprising the steps of extracting microorganisms from the filter and analysing the extracted microorganisms.

26. A method as claimed in claim 25 wherein the analysis step comprises a PCR amplification step.

27. A method as claimed in claim 25 wherein the analysis step comprises DNA/RNA and/or immunological analysis.

28. A method as claimed in claim 25 wherein the analysis step comprises culturing bacteria, viruses and/or fungi on culture media.

29. A method as claimed in any of claims 24 to 28 wherein the respiratory infection is a viral infection such as a SARs-CoV-2 (Covid-19) infection.

30. A method as claimed in any of claims 24 to 29 wherein the sampling device forms the mouthpiece on a respiratory medical device.

31. A method of sampling the exhaled breath of a human or animal patient to detect a respiratory infection comprising
sampling an exhalation breath from the patient with a sampling device as claimed in any of claims 1 to 23 and
opening the sampling device at an openable fastener on the sampling device to remove the filter from the filter chamber for analysis.

32. A method as claimed in claim 31 further comprising the steps of extracting microorganisms from the filter and analysing the extracted microorganisms.

33. A method as claimed in claim 32 wherein the analysis step comprises a PCR amplification step.

34. A method as claimed in claim 32 wherein the analysis step comprises DNA/RNA and/or immunological analysis.

35. A method as claimed in claim 32 wherein the analysis step comprises culturing bacteria, viruses and/or fungi on culture media.

36. A method as claimed in any of claims 31 to 35 wherein the respiratory infection is a viral infection such as a SARs-CoV-2 (Covid-19) infection.

37. A method as claimed in any of claims 31 to 36 wherein the sampling device forms the mouthpiece on a respiratory medical device.
